(19) United States
(12) Patent Application Publication
(10) Pub. No.: US 2026/0114735 A1
(43) Pub. Date: Apr. 30, 2026

(54) THERMAL FIELD SCANNER

(71) Applicant: HEALTH MONITORING TECHNOLOGIES, INC., Irvine, CA (US)

(72) Inventors: Philip Alan Bunker, Vista, CA (US); Luba Eugenia Diangar, Newport Beach, CA (US); Ilya Vladimirovich Dubinskiy, Irvine, CA (US)

(21) Appl. No.: 18/913,362

(22) Filed: Oct. 11, 2024

Related U.S. Application Data
(63) Continuation of application No. 16/691,388, filed on Nov. 21, 2019, now abandoned.
Application No. 16/691,388 is a continuation of application No. PCT/US2018/035165, filed on May 30, 2018.
(60) Provisional application No. 62/513,362, filed on May 31, 2017.

(51) Int. Cl. A61B 5/01; A61B 5/00

(52) U.S. Cl. CPC A61B 5/015; A61B 5/0008; A61B 5/0064; A61B 5/01; A61B 2562/0271

(57) ABSTRACT

A measurement device can be used by a patient or user to scan a tissue site to generate a thermal image of the scanned tissue site. The device can measure and collect temperature data and position data using one or more thermal and position sensors. The data can be used to generate a thermal image of the scanned tissue. The thermal image can be used to monitor various tissue sites and determine location and severity of inflammation. The thermal field scanner can help patients in avoiding formation of ulcers and other dangerous medical conditions.

### INCORPORATION BY REFERENCE TO ANY PRIORITY APPLICATIONS

Any and all applications for which a foreign or domestic priority claim is identified in the Application Data Sheet as filed with the present application, as well as applications mentioned in the specification are hereby incorporated by reference under 37 CFR 1.57.

### BACKGROUND

Inflammation is part of the complex biological response of body tissues to protect the body from infection and foreign organisms, such as bacteria and viruses. Chronic inflammation can eventually cause several diseases and conditions, including some cancers, rheumatoid arthritis, atherosclerosis, periodontitis, ulcers, hay fever, and others. Inflammation needs to be well regulated. For example, chronic inflammation can result in the development of sores, such as ulcers, on an external surface of the body can cause serious health complications. Monitoring inflammation in a patient can be helpful in diagnosing, treating, and preventing diseases and conditions. For example, monitoring a diabetic's foot on a regular basis can help avoid the formation of ulcers and other dangerous consequences. Unfortunately, known techniques for monitoring inflammation are often inconvenient to use, unreliable, or inaccurate, thus reducing compliance for patients that need it the most.

### SUMMARY

The present disclosure provides a temperature measurement system and apparatus for analyzing and generating temperature measurements for regions of a patient's body. The apparatus can measure temperature of a scanned tissue site of a patient, such as, for example, a hand, foot, head, breast, or other location. The apparatus may be referred to as a measurement device, a scanner, or thermal field scanner. The apparatus can include one or more sensors for determining positional information of the apparatus, such as linear motion, rotational displacement, and other types of positional information. A data analysis system can collect data from the one or more sensors and generates a thermal image, such as a two-dimensional (2D) or three-dimensional (3D) image, based at least in part on temperature data collected at the tissue site.

Various different types of thermal, motion, and positional sensors can be used in conjunction with the thermal field scanner. A thermal sensor can be either a contact thermal sensor or a non-contact thermal sensor, such as optical thermal sensors or far infrared (IR) thermopile. A data analysis system can be incorporated into the measurement device or be a separate, stand-alone data analysis system. In some embodiments, the data analysis system can be located in a remote location. In some embodiments, the data analysis system may be at least partially in the measurement device and in a separate computing device. The measurement device and the data analysis system can communicate via either wired or wireless communication, such as over a wireless network (e.g., Wi-Fi), using near field communication protocols (e.g.,. Bluetooth®), and/or other forms of wireless communication. The generated thermal image and/or thermal image data can be displayed on a separate display device. In some embodiments, the generated thermal image and/or thermal image data may be shared with a healthcare provider. In some embodiments, the thermal image and/or the 3D thermal image data can also be updated and shared with any network-based application.

Some embodiments of the measurement device can include a body and one or more temperature sensors. The temperature sensors can be configured to generate temperature data associated with a tissue site of a patient. The measurement device can include one or more position sensors configured to generate positional data indicative of movement and position of the apparatus. The measurement device can also include a communication interface configured to communicate the temperature data, the motion data, and the positional data to a data analysis system, wherein the data analysis system can be configured to generate a thermal image of the tissue site of the patient in response to the temperature data, the motion data, and the positional data.

In some embodiments, the one or more temperature sensors can be optical temperature sensors, infrared temperature sensors, and/or micro bolometer temperature sensors. In some embodiments, the one or more motion sensors are accelerometers, gyroscopes, and/or geomagnetic sensors. In some embodiments, the thermal image generated can be based on an average temperature and temperature gradient of the tissue site. In some embodiments, the thermal image of the tissue site can be generated based on a 3D spatial vector data, wherein the 3D spatial vector data is based on a linear motion data and Euler spatial position angle data. The linear motion data can be calculated using a relative x-y movement of the handheld temperature scanning apparatus, wherein the relative x-y movement is calculated from last known x-y coordinates of the handheld temperature scanning apparatus.

Some embodiments of generating a thermal image of a tissue site can comprise generating temperature data associated with at least a portion of the tissue site. Temperature data is generated by one or more temperature sensors disposed within the handheld temperature scanning apparatus. The method can also comprise generating, by one or more motion sensors disposed within the handheld temperature scanning apparatus, motion data and positional indicating movement and position of the apparatus. The motion data and positional data can be generated contemporaneously with the temperature data. The method can also comprise providing, through a communication interface of the handheld temperature scanning apparatus, at least a portion of the temperature data and at least a portion of the motion data to a data analysis system. The method can further comprise generating, by the data analysis system, a thermal image of the tissue site of the patient. The thermal image can comprise a (i) topography of the tissue site based at least in part on the motion data and positional data, and (ii) thermal temperature data overlaid on the topography based at least in part on the temperature data.

### BRIEF DESCRIPTION OF THE DRAWINGS

FIG. 1A illustrates an embodiment of a measurement device.

FIGS. 1B and 1C illustrate another embodiment of a measurement device.

FIG. 2 provides an embodiment of a schematic block diagram illustrating components and interactions between components of the measurement device 100 and the data analysis system 200.

FIG. 3 illustrates an embodiment of an optical temperature sensor.

FIG. 4A illustrates an embodiment of a user interface.

FIG. 4B illustrates another embodiment of a user interface.

FIG. 5A illustrates a perspective view of an embodiment of a measurement device.

FIG. 5B illustrates an exploded view of the embodiment of the measurement device of FIG. 5A.

FIG. 5C illustrates a cross-section of the embodiment of the measurement device of FIG. 5A.

FIG. 6A provides an illustration of an embodiment of a measurement device with possible rotational/angular displacements about x, y, and z-axis.

FIG. 6B provides an illustration of determining linear displacement of a measurement device along an x-y plane.

FIG. 7A illustrates an embodiment of scanning a tissue site with a measurement device.

FIG. 7B illustrates another embodiment of scanning a tissue site with a measurement device.

FIG. 8 illustrates a flowchart of an embodiment of a thermal image generation process.

### DETAILED DESCRIPTION

### Overview

The present disclosure provides embodiments of a temperature measurement system and apparatus for analyzing and generating temperature measurements for regions of a patient's body. The temperature measurement system can generate temperature measurements using a handheld measurement apparatus to scan the region of the body. The measurement apparatus can include one or more temperature sensors to acquire temperature data and one or more position sensors to acquire positional data associated with the region. A data analysis system can analyze temperature and positional data acquired by the measurement apparatus in order to generate a two dimensional (2D) or three dimensional (3D) thermal image, such as a thermogram, of the scanned area. A user can scan different regions of the body, such as the foot, leg, hand, breast, head, or other part of the body. The thermal data can be analyzed in real-time and can be output to one or more display devices and/or computing devices during use. The thermal image topography of the scanned tissue site can be generated in substantially real time using the positional and thermal data acquired by the measurement device. A completed thermal image of a region of a patient can be output for review on a user's computing device and/or may be provided to a healthcare provider.

The thermal image can permit patients, their healthcare providers, and/or their caregivers to analyze the health of a patient and help to reduce the risk of more serious complications. The temperature data can be used to evaluate and assess potential inflammation and/or other health related conditions. For example, a thermal image can help to diagnose and detect diabetic ulcers in a patient prior to emergence of an ulcer. The thermal image can be used to detect any type of inflammation of a human and/or hematothermal animal body. The measurement apparatus can be used to scan hairy, non-hairy, flat, round, and/or other 2D and 3D surfaces. In some embodiments, the measurement apparatus can be used in other industrial or cosmological applications.

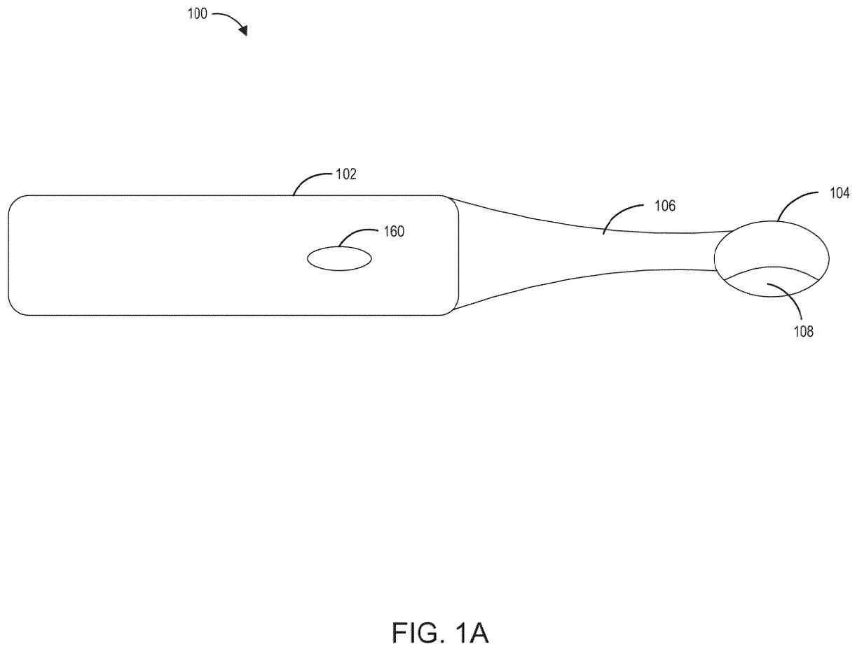

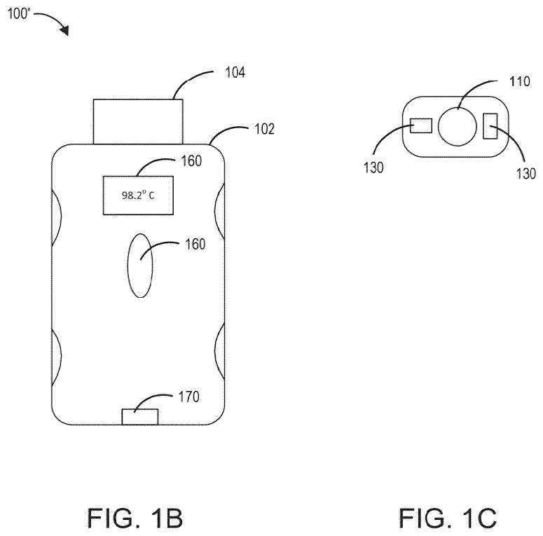

FIG. 1A illustrates an embodiment of a temperature measurement device 100 that can be used in accordance with embodiments described herein. The measurement device 100 has a wand shape comprising a body or handle portion 102 and a head 104 positioned at a distal end of an elongated body 106 from the handle portion 102. The head 104 can have any shape or size as needed. In some embodiments, the measurement device can use a larger head for non-hairy tissue sites and a smaller head for hairy tissue sites. The smaller head can allow for the measurement of the tissue site underneath the hair. The head can have a sensor interface portion 108 disposed on at least one side of the head. The sensor interface portion 108 can be configured to house one or more temperature sensors 110. For optical temperature sensors, it may be desirable for the sensors to be offset from the skin of the patient. The sensor interface portion 108 can be configured to offset the sensors at a defined distance from the skin of a patient. In some embodiments, the sensor interface portion 108 may include a clear cover that offsets the sensors from the outer surface of the head 104. In some embodiments, the sensor interface portion 108 can include a cavity, and the sensors 110 can be positioned within the cavity, which may or may not include a cover portion. In some embodiments, the sensor interface portion 108 may include one or more standoffs to offset the sensors. In some embodiments, the sensors may be positioned adjacent to the skin in order to generate a temperature measurement.

The device 100 includes one or more motion sensors 130. The motion sensors 130 can be disposed within the various portions of the device 100. For example, motion sensors 130 can be disposed within the handle portion 102, the elongated portion 106, and/or the head portion 104. The head 104 can have a fixed orientation. In some embodiments, the head 104 can be configured to swivel relative to the body 102 and/or the elongated portion 106. The temperature sensors 110 and motion sensors 130 will be described in more detail below. The head can be configured so that it is easy for the patient to keep the sensors substantially perpendicular to the tissue site during scanning.

A patient/user activates the device using a patient I/O interface 160. The I/O interface 160 can be a user actuatable interface such as a button and/or other interface elements. A user/patient may scan a selected region of the body in order to generate the thermal scan of the area. The scanned data can be provided to a data analysis system 200. The data analysis system can process and output the thermal scan data for review by the user/patient and/or other individuals, such as a healthcare provider.

FIGS. 1B and 1C illustrates another embodiment of a temperature measurement device 100′ that can be used in accordance with embodiments described herein. FIG. 1B illustrates a top view of the measurement device 100′ and FIG. 1C illustrates a detail view of the end of the head portion 104. The measurement device 100′ has a body or handle portion 102 and a head 104 positioned at a distal end of the handle portion 102. The device 100′ includes one or more motion sensors 130 (e.g., an optical motion sensor and a positional sensor) and a temperature sensor (e.g., an IR temperature sensor). In this embodiment, the motion sensors 130 can be disposed within the head portion 104. A patient/user can activate the device 100′ using a patient I/O interface 160. The temperature measurement device 100′ can include a display element 162 indicating state of the device and/or other information (e.g., current temperature reading). The device 100′ includes a physical device I/O interface 170 (e.g., a universal serial bus (USB) port).

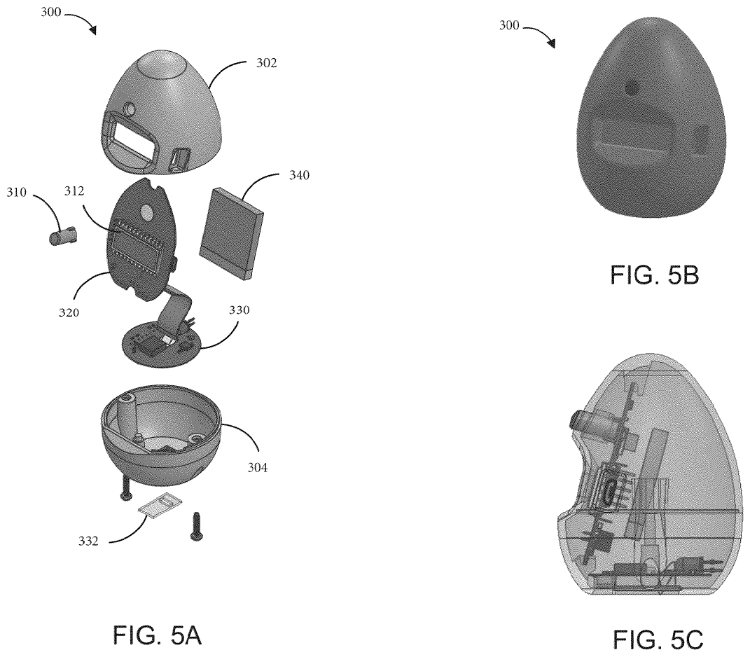

FIG. 5A illustrates perspective view of another embodiment of a temperature measurement device 300 that can be used in accordance with embodiments described herein. FIG. 5B illustrates an exploded view of the temperature measurement device 300 shown in FIG. 5A. FIG. 5C illustrates a cross-section of the temperature measurement device 300 shown in FIG. 5A. The temperature measurement device 300 has a housing including a top portion 302 and a bottom portion 304 that define the outer structure of the temperature measurement device 300. The top portion 302 can be mechanically coupled to the bottom portion 304. In some embodiments, the top portion 302 and the bottom portion 304 have corresponding threads that allow the portions to be removably connected to one another. In some embodiments, the top portion 302 and the bottom portion 304 can be coupled using one or more fasteners. In some embodiments, a latch mechanism can be used to couple the top portion 302 with the bottom portion 304.

The temperature measurement device 300 includes a patient I/O interface 310, a display element 312, a printed circuit board assembly 320, a sensor 330, a sensor window 332, and a battery 340. The patient I/O interface 310, the display element 312, and the sensor 330 are operatively connected to the printed circuit board assembly 320. The battery 340 is also operatively connected to the printed circuit board assembly 320. The sensor window 332 is mechanically connected to the bottom surface of the bottom portion 304. The sensor window 332 can be transparent or partially transparent to allow light from the sensor 330 to pass through the sensor window 332. In some embodiments, the sensor window 332 may be tinted a different color. A patient/user can activate the temperature measurement device 300 using the patient I/O interface 310. The display element 312 can indicate one or more states of the device 300 and/or other information (e.g., current temperature reading). The device 300 can include a physical device I/O interface (e.g., a universal serial bus (USB) port).

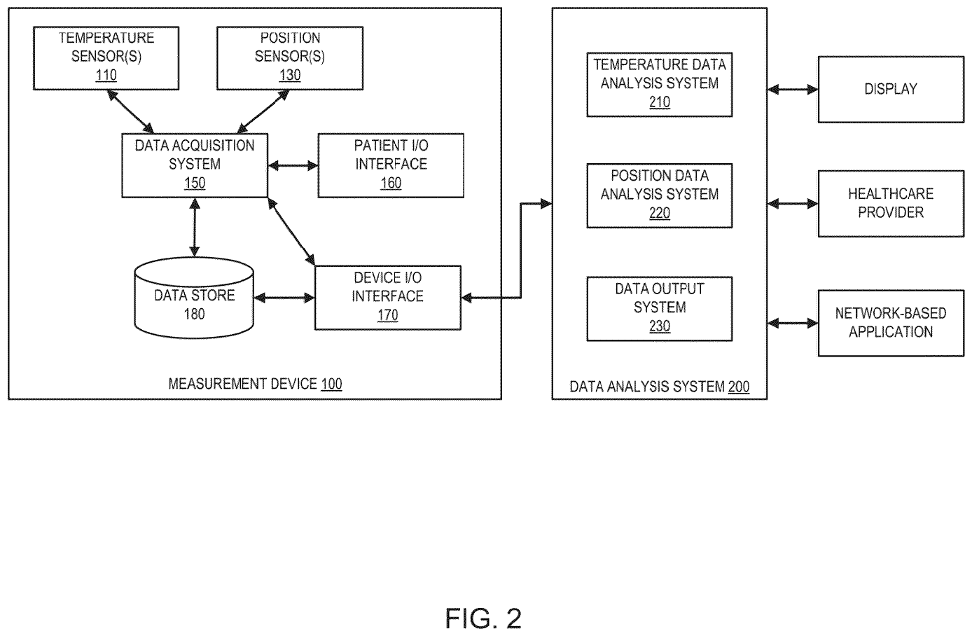

FIG. 2 provides a schematic block diagram illustrating components and interactions between components of the measurement device 100 and the data analysis system 200. For purposes of the illustrated embodiment, however, the illustration has been simplified such that many of the components utilized to facilitate operation of the various systems are not shown. One skilled in the relevant art will appreciate that such components can be utilized and that additional interactions would accordingly occur without departing from the spirit and scope of the present disclosure.

### Measurement Device

The measurement device 100 can include, among other components, at least one temperature sensor 110, at least one motion sensor 130, a data acquisition system 150, a patient I/O interface, a device I/O interface, and at least one data store 180.

### Temperature Sensors

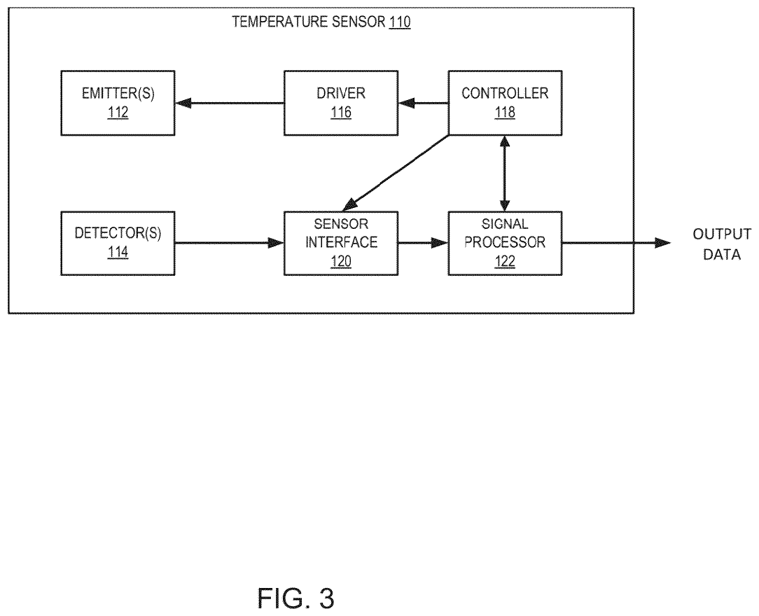

The one or more temperature sensors 110 can be fixed in the measurement device to provide temperature data for a scanned tissue site of a patient. In some embodiments, a plurality of temperature sensors can be arranged in a line or in an array or matrix of temperature sensors 110 and fixed in place within the measurement device 110. The temperature sensors 110 can be positioned on a printed circuit board within the device 100. The pitch or distance between the temperature sensors can be relatively small, thus permitting more temperature sensors 110 for conducting temperature measurements. Preferably, the temperature sensors are non-contact temperature sensors, such as optical temperature sensors (e.g., an infrared temperature sensor or micro bolometer sensor). An exemplary embodiment of an optical temperature sensor is described and illustrated with respect to FIG. 3.

In some embodiments, the temperature sensors 110 may include temperature sensitive resistors (e.g., printed or discrete components mounted onto the device 100), thermocouples, fiber optic temperature sensors, or a thermochromic film. Accordingly, when used with temperature sensors 110 that require direct contact, illustrative embodiments may use a cover on the device 100 having a thin material with a relatively high thermal conductivity.

As discussed herein, regardless of their specific type, the temperature sensors 110 generate a plurality of corresponding temperature data values for a plurality of portions/spots during scanning of the tissue site of the patient. Additional sensors can provide additional redundant data that may be helpful in filtering out anomalous or bad temperature data.

### Position Sensors

The position sensors 130 can be configured to generate movement and/or positional data indicative of position and/or movement of the measurement device 100. The motion/position sensors 130 can gather and output data associated with the position and/or motion of the measurement device 100, such as, for example, an accelerometer, a magnetometer, a gyroscope, and/or other sensors. In some embodiments, the position sensors can include an optical sensor, such as sensor on a computer mouse. The sensors are configured to detect and output indications of position and/or movement of the device 100. The indication of position or movement can be output from the sensor in various forms, which can be dependent on the specific sensor being used. Some examples of sensor outputs can include analog outputs (e.g., varying voltage levels that fluctuate between a ground voltage and a supply voltage level), digital outputs (e.g., discrete output values), wave forms (e.g., pulse-width modulation (PWM) output square waves with a known period, but a varying duty cycle), and the like. The data corresponding to the indication of position or movement can be stored for further processing. The output data from the sensors may be processed using one or more hardware and/or software-based filters prior to storage. For example, the signals may be filtered to remove or reduce signal noise. The motion and positional data analysis system 220 can perform additional post-processing, analysis, and output on the motion and position data. In some embodiments, one or more sensors 130 may be combined with a microcontroller to generate and output positional and motion data. For example the sensors may be able to output rotation, linear acceleration, gravity, heading, and other positional and/or motion related output data.

### Data Acquisition System

The data acquisition system 150 can be configured to interface and communicate with the temperature sensors 110 and the motion sensors 130. The data acquisition system 150 can aggregate the sensor data and provide the sensor data to one more data stores 180 for storage. The data acquisition system 150 can communicate with the patient I/O interface 160 to receive instruction regarding the operation of the temperature sensors 110 and/or motion sensors 130. The data acquisition system 150 can provide instructions to the temperature sensors 110 and/or motion sensors 130 to initiate collection of data for a defined period of time. The data acquisition system 150 can provide instructions to the temperature sensors 110 and/or motion sensors 130 to deactivate and cease the collection of data. The data acquisition system can be configured to communicate with the device I/O interface to engage in transfer of the stored data to the data analysis system 200.

### Patient I/O Interface

The patient I/O interface 160 can provide an interface for a patient to control the device 100. The interface can include one or more inputs for controlling operation of the sensors on the device. For example, the device may have a button, such as illustrated in FIG. 1, that initiates the scanning functionality. The patient I/O interface 160 can provide a user-friendly experience for the patient. In some embodiments, the patient I/O interface 160 may include a display, such as an LED display that can provide instructions or information to the patient operating the device. For example, the display may indicate whether the client is moving the device too fast or too slow.

### Device I/O Interface

The device I/O interface 170, which may be controlled by the data acquisition system 150 and other electronics on the device 100 can selectively transmit or forward the acquired data from the data store 180 to the data analysis system 200 on a remote computing device. In some embodiments, the device I/O interface 170 can be physical port interface, such as a universal serial bus (USB) interface or other type of physical interface that can electrically couple to a computing system using a physical connection. In some embodiments, the device I/O interface 170 can be a wireless connection that can communicate using wireless communication protocols and technologies, such as for example, near field communication (e.g., Bluetooth®), Wi-Fi, or other wireless transmission technologies. Protocols and components for communicating via the Internet or any of the other aforementioned types of communication networks are well known to those skilled in the art of computer communications and thus, need not be described in more detail herein.

### Data Store

The one or more data stores 180 are configured to store sensor data generated by the temperature sensors 110 and the motion sensors 130. The data store 180 can be a tangible storage medium configured to store the sensor data. The data store 180 can be a volatile or nonvolatile storage medium, such as a hard drive, high-speed random-access-memory (“RAM”), or solid-state memory. The data store 180 can communicate with the data acquisition system to receive data for storage and provide data to the device I/O interface for transmittal to the data analysis system.

### Data Analysis System

The data analysis system can correspond to computer readable instructions executable on one or more computing devices. The computer readable instructions can be configured as a software application or module comprising the computer readable instructions. In general the software application refers to logic embodied in hardware or firmware, or to a collection of software instructions stored on a non-transitory, tangible computer-readable medium, possibly having entry and exit points, written in a programming language, such as, for example, C, C++, C #, or Java. A software application or module may be compiled and linked into an executable program, installed in a dynamic link library, or may be written in an interpreted programming language such as, for example, BASIC, Perl, or Python. It will be appreciated that software applications and/or modules may be callable from other modules or from themselves, and/or may be invoked in response to detected events or interrupts. Software modules may be stored in any type of computer-readable medium, such as a memory device (e.g., random access, flash memory, and the like), firmware (e.g., an EPROM), or any other storage medium. The software modules may be configured for execution by one or more CPUs in order to cause data analysis system 200 to perform particular operations. Generally, the modules described herein refer to logical modules that may be combined with other modules or divided into sub-modules despite their physical organization or storage.

As stated, the data analysis system 200 can operate on one or more computing devices. The computing devices can correspond to a wide variety of devices or components that are capable of initiating, receiving or facilitating operations and communications with the measurement device 100 and over one or more communication networks including, but not limited to, personal computing devices, electronic book readers (e.g., e-book readers), hand held computing devices, integrated components for inclusion in computing devices, cellular telephones, smart phones, personal digital assistants, laptop computers, gaming devices, media devices, and the like. In an illustrative embodiment, the computing devices can include a wide variety of software and hardware components.

The data analysis system 200 can include, among other components, a temperature data analysis system 210, a motion data analysis system 220, and a data display output system 230.

### Temperature Data Analysis System

The temperature data analysis system 210 is configured to analyze the data in order to form a thermal scan of the scanned tissue site of the patient based on a plurality of temperature readings. The temperature data analysis system 210 can use conventional techniques to extrapolate data from the real temperature data in order to approximate the temperature at each point of the tissue site. The temperature data analysis system 210 embodiment may be configured to acquire temperature data for the various data points in a small amount of time, such as multiple measurements per second, or less frequently, such as every one to three seconds.

The temperature sensors 110 can provide redundant temperature data that can be analyzed and used to filter out erroneous data points. Ultimately, the temperature data is used to generate a thermal image of the scanned tissue site. The thermal image may be a two dimensional or 3D image of the tissue site.

The temperature data analysis system 210 can compute a temperature gradient of the scanned tissue site based on a determined temperature of the tissue site, such as an average temperature, a median temperature, or other temperature value determined using a defied algorithm. The determined temperature of the tissue site can be based on at least a portion of the temperature measurements for the tissue site. In some embodiments, the determined temperature may be based on substantially all of the temperature data collected for a tissue site. The temperature data analysis system 210 can use the determined temperature of the tissue site to determine temperature gradient for the thermal image. The color coding for the thermal image can be based on the difference in temperature from the determined temperature. Different colors can be associated with different groupings of temperatures relative to the determined temperature. Using this methodology, each scanned tissue site can have a different temperature gradient that is used for color coding the thermal image. In some embodiments, the thermal image output can include the change in temperature relative to the determined temperature and the absolute temperature.

### Motion and Positional Data Analysis System

The motion and positional data analysis system 220 can be configured to process and analyze the movement and positional data associated with the motion/position sensors 130. The motion and positional data analysis system 220 can track and analyze the movement data over time in order to generate a 3D model of the scanned tissue site. The motion and positional data analysis system 220 can be configured to record position, velocity, orientation, rotations, and other position based measurements received from the motion sensors. The movement data can include acceleration data and orientation data that can be used to analyze various aspects of the tissue site scanning. The system can analyze the collected data (e.g., acceleration data, orientation data, time-based data, etc.) in order to determine the topography of the tissue site. The motion and positional sensor data in conjunction with the temperature data can be used to determine when the device is not positioned over a portion of the tissue site. The motion data analysis system 220 can algorithmically determine the topography of the tissue site using various techniques, such as for example, synthetic aperture averaging.

In some embodiments, the position data analysis system 220 incorporates a planar scanner for linear motion which maps X-Y coordinates in a relative or delta-based analysis. In some embodiments, the planar scanner may not measure absolute position. It may measure relative X-Y movement based on the last known X-Y position. If the sensor is removed from the surface while scanning, the X-Y position information may be discarded. If a temperature measurement device 100 is deactivated, such that scanning is paused, the motion data analysis system 220 can use the last known X-Y position once the sensor is reactivated, or the position data can be reset.

In some embodiments, the orientation of the temperature measurement device 100 can be determined using one or more positional sensors, such as accelerometers, gyroscopes, geometric sensors, and/or a combination of the sensors. These sensors may be used in conjunction with an advanced micro electrical mechanical system (MEMS) device to output Euler spatial position angles, which can be used to determine spatial coordination of the temperature measurement device 100. 3D spatial vectors can also be mapped by using the Euler spatial position angles in conjunction with linear motion sensor. In one embodiment, the temperature can be captured by non-contact far IR thermopile, which can have an accuracy of 0.02 degrees and a sample rate of about 1k samples per second. In some embodiments, the temperature can be captured by contact thermal sensors. It should be appreciated that greater accuracy and higher sample rate will lead to generating better 3D model of a scanned tissue site.

The data measured and collected by the temperature measurement device 100 and sent to the data analysis system 200 can include at least some of the following data types. Delta X values and Delta Y values can indicate differences in x-y coordinates before and after movement by the temperature measurement device 100, wherein x-y coordinates can represent the coordinates of the temperature measurement device 100 with respect to a reference point. In some embodiments, the planar scanner can have varying level of sensitivity, represented in counters per inch (CPI) or dots per inch (DPI). For example, in one embodiment, the planar scanner can collect and measure at about 800 CPI. In some embodiments, the planar scanner can collect and measure between 500 and 1,000 CPI. It will be understood by those skilled in the art that a planar scanner with higher CPI (or DPI) will yield more accurate locational measurement of the temperature measurement device 100.

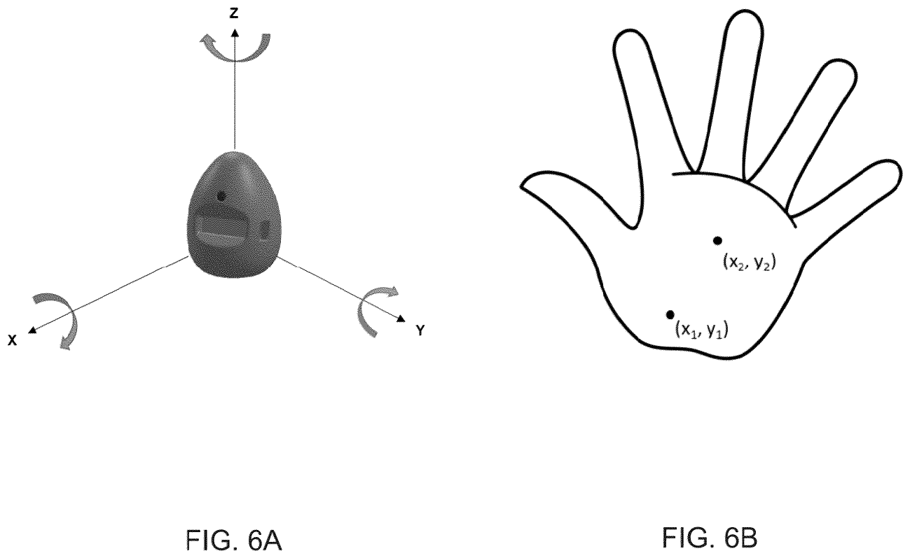

FIG. 6B shows an example of determining Delta X and Delta Y for the temperature measurement device 100. The temperature measurement device 100 is initially positioned at a first location having x-y coordinates of (X1, Y1). When the temperature measurement device 100 is moved to a second location having x-y coordinates of (X2, Y2), data acquisition system 150 of the temperature measurement device 100 collects the x-y coordinates of the first and the second location. The data for the coordinates is then sent to motion data analysis system 220 of the data analysis system 200, and the motion data analysis system 220 determines the Delta X and Delta Y corresponding to the movement from the first location to the second location.

The temperature measurement device 100 can use one or more positional sensors, such as accelerometers, gyroscopes, geometric sensors, and/or a combination of the sensors to detect and measure angular measurements in addition to linear measurements. The angular measurements may include heading, roll, and pitch. It will be understood by those skilled in the art that the angular measurements—roll, pitch, and heading—-each describe rotational movement of an object (e.g., the temperature measurement device 100) about x, y, and z-axis, respectively. The angular measurements can measure and detect a rotational/angular displacement between about 0.001 and 0.01 degrees, about 0.01 and 0.05 degrees, about 0.05 and 0.1 degrees, or about 0.001 degrees, 0.015 degrees, 0.002 degrees, 0.025 degrees, 0.03 degrees, 0.035 degrees, 0.04 degrees, 0.045 degrees, 0.05 degrees, 0.055 degrees, 0.06 degrees, 0.065 degrees, 0.07 degrees, 0.075 degrees, 0.08 degrees, 0.085 degrees, 0.09 degrees, 0.095 degrees, 0.1 degrees, 0.2 degrees, 0.3 degrees, 0.4 degrees, 0.5 degrees, or ranges including any two of the aforementioned values. FIG. 6A depicts an exemplary temperature measurement device 100 with its x, y, and z-axis. Roll, pitch, and heading can represent rotational/angular displacement of the temperature measurement device 100 along its x, y, and z-axis.

The temperature measurement device 100 can also measure and collect temperature data using one or more temperature sensors 110. In some embodiments, the temperature sensors 110 can measure and detect a temperature change of 0.1 degrees Kelvin. In some embodiments, the temperature sensors 110 can detect and measure a temperature change of 0.05 degrees Kelvin. In some embodiments, the temperature sensors 110 can detect and measure a temperature change between about 0.01 and 0.05 degrees, about 0.05 and 0.1 degrees, about 0.1 and 0.5 degrees, or about 0.01 degrees, 0.02 degrees, 0.03 degrees, 0.04 degrees, 0.05 degrees, 0.06 degrees, 0.07 degrees, 0.08 degrees, 0.09 degrees, 0.1 degrees, 0.2 degrees, 0.3 degrees, 0.4 degrees, 0.5 degrees, or ranges including any two of the aforementioned values.

In some embodiments, there can be a recommended scanning speed for generating an adequate 3D image of a tissue area. It should be appreciated to those skilled in the art that a scanning speed too great can result in poor 3D image of the scanned tissue area. Likewise, it should also be appreciate to those skilled in the art that slower scanning speed can result in better 3D image of the scanned tissue area.

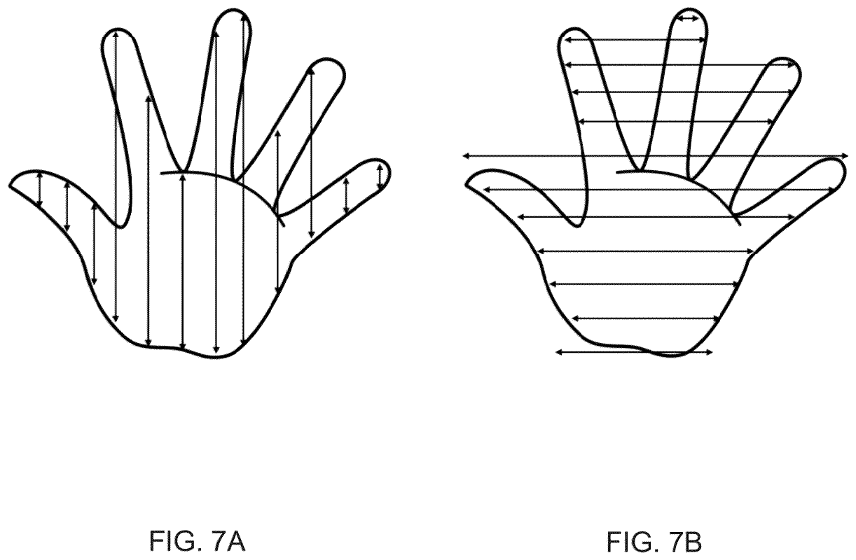

FIGS. 7A and 7B show an exemplary method of scanning a tissue area. For example, the temperature measurement device 100 can be moved in a first direction and back to cover the entire tissue area to be scanned. The temperature measurement device 100 then can be moved in a second direction that is substantially orthogonal to the first direction. Such method ensures that the entire tissue area is scanned, and provides additional motion and temperature data.

The data analysis system 200 can synchronize all of the sensors (e.g., temperature sensor, accelerometers, gyroscopes, geometric sensors, planar scanner, etc.). The data analysis system 200 then can collect data from the synchronized sensors at a rate of 30 samples per second. In some embodiments, the data analysis system 200 can collect data at a rate between about 5 and 20 samples per second, about 20 and 40 samples per second, about 40 and 60 samples per second, about 60 and 80 samples per second, or about 5 samples per second, 10 samples per second, 15 samples per second, 20 samples per second, 25 samples per second, 30 samples per second, 35 samples per second, 40 samples per second, 45 samples per second, 50 samples per second, 55 samples per second, 60 samples per second, 65 samples per second, 70 samples per second, 75 samples per second, 80 samples per second, 85 samples per second, 90 samples per second, 95 samples per second, 100 samples per second, or ranges including any two of the aforementioned values. The data analysis system 200 can then use the collected data to generate data packets based on the collected data. In some embodiments, the data packets can include raw data from the sensors. In some embodiments, the data packets can include bits converted from the raw data.

The data analysis system 200 then can transfer the data packets to a host for processing. The host and the data analysis system 200 of the temperature measurement device 100 can establish communication either with wire or wirelessly. In some embodiments, the host and the temperature measurement device 100 can communicate via wireless communication protocols and technologies, such as for example, near field communication (e.g., Bluetooth®), Wi-Fi, or other wireless transmission technologies. The data packets can be transferred to the host at a data rate of 19.2 kbps. In some other embodiments, the data rate can be about between 5 kbps and 10 kbps, about 5 kbps and 100 kbps, 50 kbps and 500 kbps, 100 kbps and 1 mbps, 500 kbps and 2 mbps, 1 mbps and 1 gbps, or ranges including any two of the aforementioned values. In some embodiments, the host confirms reception of the data packet from the temperature measurement device 100.

In some embodiments, a tissue site can be scanned multiple times to generate a 3D model of the scanned tissue site (e.g., hand). As noted above, the motion data analysis system 220 can record position, velocity, orientation, rotations, and other position based measurements received from the motion sensors 130. The motion data analysis system 220 can gather the movement data from a first scan and generate a first 3D model of the tissue site. The motion data analysis system 220 can then use the first 3D model of the tissue site for a second, subsequent scan. The motion data analysis system 220 can then generate a second 3D model of the tissue site using the movement data collected from the second scan. In some embodiments, a user interface can show each iteration of the 3D model of the scanned tissue site after each scan. In some embodiments, the user interface can show the 3D model of the tissue site after the last scan. In some embodiments, regions that are not tissue sites (such as, spaces between fingers) can be automatically filtered out.

### Data Output System

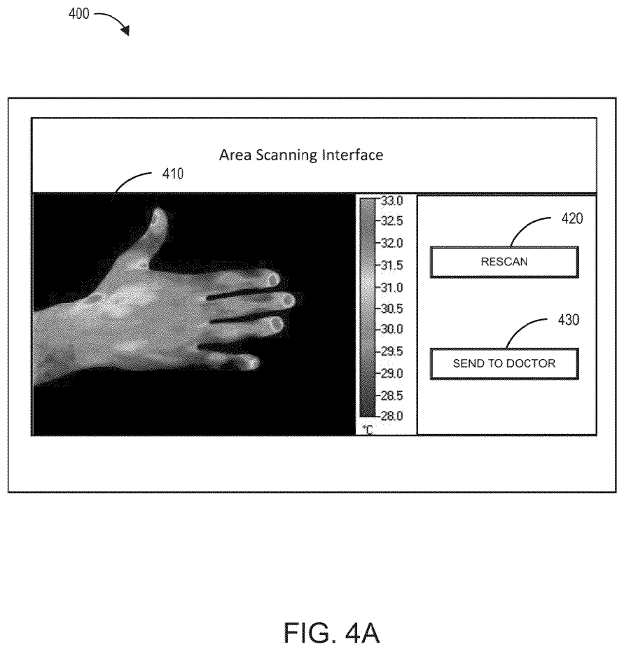

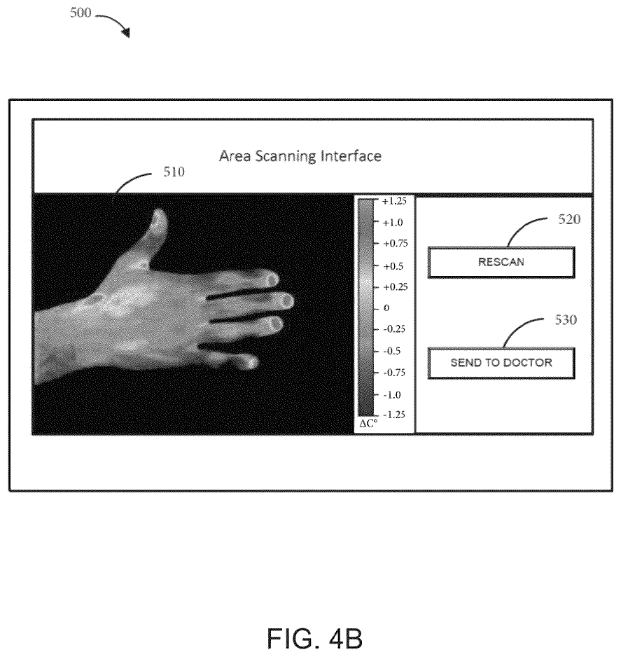

The data output system 230 can be configured to generate graphical display instructions for outputting the thermal scan to one or more outputs. An example embodiment of a user interface is illustrated in FIG. 4. The data output system can provide instructions for the display of the thermal scan to a display, a healthcare provider, a network-base application, and/or other output capable of displaying the thermal scan. The data output system 230 can provide real time updates to the thermal scan within the user interface. For example, the thermal scan can indicate regions of the tissue site that do not have sufficient data to generate an image of the thermal scan. In this manner, the patient can easily scan the area of the tissue site to complete the thermal scan.

### Example Temperature Sensor

FIG. 3 illustrates an embodiment of an optical temperature sensor, such as an infrared temperature sensor, which can measures optically measure temperature of a surface. A temperature sensor 110 can generate a sensor signal that can be converted to a numerical output of a temperature at a tissue site of a patient. The temperature sensor can detect surface temperature at the selected tissue site on a patient, such as foot, leg, hand, breast, head, or other part of the body. The drivers 116 activate the emitters 112 according to instructions received the controller 118 such as an optical light that can indicate the relative location of the temperature reading. The sensor interface 120 conditions and digitizes the sensor signal received from the detector 114. The signal processor 122 inputs the conditioned and digitized sensor signal received from the sensor interface 120 and calculates a temperature of the selected tissue site. The signal processor 122 can provide a numerical output of a temperature at the tissue site. In some embodiments, a temperature sensor can generate temperature measurements at time-based intervals depending on the usage and/or hardware of the sensor. For example, a sensor may generate measurements four times per second, two times per second, or at another interval.

### Example User Interface

FIGS. 4A and 4B depict an example embodiment of a user interface 400 and a user interface 500. The user interface 400 and the user interface 500 shown includes one or more user interface controls that can be selected by a user, for example, the user interface may be generated in a software application, on a mobile app, using a browser, and/or other application software. The user interface controls shown are merely illustrative examples and can be varied in other embodiments. For instance, buttons, dropdown boxes, select boxes, text boxes, check boxes, slider controls, and other user interface controls shown may be substituted with other types of user interface controls that provide the same or similar functionality. Further, user interface controls may be combined or divided into other sets of user interface controls such that similar functionality or the same functionality may be provided with very different looking user interfaces. Moreover, each of the user interface controls may be selected by a user using one or more input options, such as a mouse, touch screen input, or keyboard input, among other user interface input options.

In the example embodiment of the user interface 400, the interface includes a thermal scan area 410 that provides a real-time image of the scanned tissue site during run-time usage of the scanner. The scanned tissue site may be a hand, a foot, a head, or any other body part. The thermal scan area 410 can comprise a real-time image of the scanned area depicted in different spectrum of colors or shades of colors to illustrate a different temperatures measured in the scanned area. For example, relatively colder areas can be represented in colder colors (e.g., blue or navy), whereas relatively warmer areas can be represented in warmer colors (e.g., red or pink). Different colors or spectrums of colors can be used to represent different temperature readings in the scanned area. For example, as shown in FIG. 4A, the temperature readings measured for a hand may range between 28 degrees Celsius and 34 degrees Celsius. For example, a color spectrum ranging between navy and pink can be used to represent different temperatures readings of the scanned tissue site. By associating different spectrum of colors to different temperature readings, the thermal scan area 410 can visually indicate areas associated with low or high temperature readings.

In some embodiments, the image may be output to the screen after processing the temperature and motion data from the thermal scanner. In the illustrated embodiment, the scan has been completed. Prior to completion of the scan, the image may include blank areas that have not been scanned or do not have sufficient data to generate an image. The user interface may include a number of user interface controls, such as a rescan control 420 for initiating a new scan of the same or a different tissue site, or a transmit control 430 for transmitting the scan data to a healthcare provider. The interface may include any number of controls and/or interface option for controlling the thermal scan output associated with the date and is not limited by the illustrated embodiments.

In the example embodiment of the user interface 500, the interface includes a thermal scan area 510 that provides a real-time image of the scanned tissue site during run-time usage of the scanner. The scanned tissue site may be a hand, a foot, a head, or any other body parts. The thermal scan area 510 can comprise a real-time image of the scanned area depicted in different spectrum of colors or shades of colors to illustrate a different temperature gradients measured in the scanned area. The temperature measurement device 100 can collect temperature readings from an area-of-interest within the scanned area. The data analysis system 200 then can use the temperature readings to determine an average temperature of the area-of-interest. The data analysis system 200 can then calculate a temperature gradient for various points within the area-of-interest using real-time temperature readings and the calculated determined temperature (such as, an average temperature). In some embodiments, the determined temperature of the area-of-interest can be determined automatically. In some embodiments, the average temperature of the area-of-interest can be determined manually. In some embodiments, the data analysis system 200 can require certain number of temperature data points to determine the average temperature.

As shown in FIG. 4B, relatively colder areas can be represented in colder colors, whereas relatively warmer areas can be represented in warmer colors. Different colors or spectrums of colors can be used to represent different temperature gradient readings in the scanned area. For example, a color spectrum ranging between navy and pink can be used to represent different temperature gradient readings of the scanned area. Warmer colors (e.g., pink or red) can be used to represent greater positive temperature gradient whereas colder colors (e.g., navy or blue) can be used to represent greater negative temperature gradient. A spectrum of colors close to the color green can be used to represent temperature gradient that is substantially close to zero.

In some embodiments, the image may be output to the screen after processing the temperature and motion data from the thermal scanner. In the illustrated embodiment, the scan has been completed. Prior to completion of the scan, the image may include blank areas that have not been scanned or do not have sufficient data to generate an image. The user interface may include a number of user interface controls, such as a rescan control 420 for initiating a new scan of the same or a different tissue site, or a transmit control 430 for transmitting the scan data to a healthcare provider. The interface may include any number of controls and/or interface option for controlling the thermal scan output associated with the date and is not limited by the illustrated embodiments.

### Thermal Image Generation Process

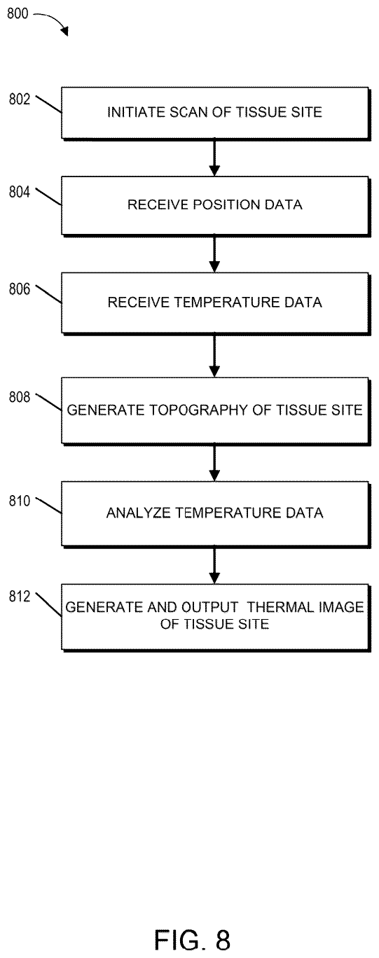

FIG. 8 illustrates an embodiment of a flowchart for a process for thermal image generation. The process 800 can be implemented by computing systems that can receive measurement data, such as position and temperature data, analyze the measurement data, and generate a thermal image. For example, the process 800, in whole or in part, can be implemented by the measurement device 100, the data analysis system 200, or other computing system. Although any number of systems, in whole or in part, can implement the process 800, to simplify discussion, the process 800 will be described with respect to the data analysis system 200 and measurement device 100.

At block 802, the data analysis system 200 or the measurement device 100 can initiate a scan of a tissue site of the patient. The measurement device may be in remote communication with the data analysis system can initiate the scan based on a signal from the data analysis system or visa-versa.

At block 804, the data analysis system 200 can receive position data generated during the scan of the tissue site. The position data can be generated by one or more positional sensors housed in the measurement device 100. The position data can be collected by the sensors at a defined rate. The position data may be stored locally on the measurement device prior to be provided to the data analysis system. In some embodiments, the position data may be provided to the data analysis system in substantially real-time during the scan of the tissue site. The position data can be generated by movement of the measurement device over the tissue area.

At block 806, the data analysis system 200 can receive temperature data generated during the scan of the tissue site. The temperature data can be generated by one or more temperature sensors housed in the measurement device 100. The temperature data can be collected by the temperature sensors at a defined rate. The temperature data may be stored locally on the measurement device prior to be provided to the data analysis system. In some embodiments, the temperature data may be provided to the data analysis system in substantially real-time during the scan of the tissue site.

At block 808, the data analysis system can generate a topography of the tissue site based at least in part on the position data. The topography of the tissue site may be a 2D or 3D topography of the tissue site. In some embodiments, the temperature data can be used to help generate the topography. For example, the temperature data may be used to filter out locations of the scan where there is no tissue present, such as in between fingers. The motion data analysis system 220 can algorithmically determine the topography of the tissue site using various techniques, such as for example, synthetic aperture averaging.

At block 810, the data analysis system can analyze the temperature data based on one or more algorithms. The data analysis system 200 can use the temperature data to determine a calculated temperature value associated with the tissue site. The data analysis system 200 can calculate a temperature gradient for various points within the area-of-interest using real-time temperature readings and the calculated determined temperature (such as, an average temperature).

At block 812, the data analysis system can generate and output a thermal image after processing the position and temperature data. The generated thermal image can be 2D or 3D and can be output to one or more computing systems. For example, the thermal image may be output to a device associated with the patient, such as a tablet computing device, and may be provided to a network based computing system such that a remote healthcare professional can access the data.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more general purpose computers or processors. The code modules may be stored in any type of non-transitory computer-readable medium or other computer storage device. Some or all the methods may alternatively be embodied in specialized computer hardware. In addition, the components referred to herein may be implemented in hardware, software, firmware or a combination thereof.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1-20. (canceled)

21. A handheld temperature scanning apparatus comprising:
a body including:
one or more temperature sensors configured to generate temperature data associated with a tissue site of a patient, wherein the one or more temperature sensors are configured to measure and detect a temperature change of at least 0.01 degrees Kelvin;
one or more position sensors configured to generate positional data indicative of linear and angular displacement of the apparatus, wherein the one or more position sensors are configured to determine position, velocity, orientation, acceleration, and rotation data including heading, pitch, and roll, wherein the sensors are configured to determine angular displacement of at least 0.001 degrees;
a communication interface configured to communicate the temperature data and the positional data to a data analysis system, the data analysis system configured to:
synchronize measurements of the one or more temperature sensors and the one or more position sensors during a scan;
collect synchronized measurements at a rate of at least 30 samples per second during the scan;
filter locations of the scan where no tissue is present based on the temperature data;
determine a three dimensional (3D) topography of the tissue site of the patient;
generate a thermal image of the tissue site of the patient based at least in part on the temperature data and the positional data, wherein the thermal image comprises a thermal temperature data overlay on the 3D topography, wherein the data analysis system is configured to generate the thermal image of the tissue site by:
determining an average temperature of the tissue site;
determining a temperature gradient for the tissue site based at least in part on the determined average temperature of the tissue site; and
determining an output color for each portion of the thermal image based at least in part on the temperature data and the determined temperature gradient, wherein the output color for each portion of the thermal image is based on the difference in temperature compared to the determined average temperature; and
generate instructions to output the thermal image on a display in real-time.

22. The apparatus of claim 21, wherein the body further comprises a head disposed at a distal end of the body, wherein the one or more temperature sensors are disposed within the head.

23. The apparatus of claim 21, wherein the one or more temperature sensors are optical temperature sensors, infrared temperature sensors or micro bolometer temperature sensors.

24. The apparatus of claim 21, wherein the one or more position sensors are accelerometers or gyroscopes.

25. The apparatus of claim 21, wherein the temperature data includes temperature values generated at discrete points on the tissue site.

26. The apparatus of claim 21, wherein the thermal image is generated based at least in part on three-dimensional spatial vector data comprising linear motion data and Euler spatial position angle data.

27. The apparatus of claim 26, wherein the linear motion data is based on a relative x-y movement of the handheld temperature scanning apparatus.

28. The apparatus of claim 26, wherein the data analysis system is configured to determine the 3D topography using synthetic aperture averaging.

29. A method of generating a three-dimensional (3D) thermal image of a tissue site of a patient, the method comprising:
generating, by one or more temperature sensors disposed within a handheld temperature scanning apparatus, temperature data associated with at least a portion of a tissue site of a patient;
generating, by one or more position sensors disposed within the handheld temperature scanning apparatus, positional data indicating movement and position of the apparatus, wherein the positional data is generated contemporaneously with the temperature data;
providing, through a communication interface of the handheld temperature scanning apparatus, at least a portion of the temperature data and at least a portion of the positional data to a data analysis system; and
generating, by the data analysis system, a thermal image of the tissue site of the patient, wherein the thermal image comprises a (i) topography of the tissue site based at least in part on the positional data, and (ii) thermal temperature data overlaid on the topography based at least in part on the temperature data,
wherein generating the thermal image of the tissue site comprises:
determining an average temperature of the tissue site;
determining a temperature gradient for the tissue site based at least in part on the determined average temperature of the tissue site;
determining an output color for each portion of the thermal image based at least in part on the temperature data and the determined temperature gradient, wherein the output color for each portion of the thermal image is based on the difference in temperature compared to the determined average temperature;
generating instructions to output the thermal image on a display in real-time.

30. The method of claim 29, wherein the communication interface is a wireless communication interface.

31. The method of claim 29, wherein the one or more temperature sensors are optical temperature sensors, infrared temperature sensors or micro bolometer temperature sensors.

32. The method of claim 29, wherein the one or more position sensors are accelerometers.

33. The method of claim 29, wherein the one or more position sensors are gyroscopes.

34. The method of claim 29, wherein the display is associated with the patient, and further comprising outputting the thermal image to a remote computing system associated with a healthcare professional.

35. The method of claim 29, wherein the thermal image includes the change in temperature relative to average temperature for the tissue site and the absolute temperature of the tissue site.
